(19) United States
(12) Patent Application Publication
(10) Pub. No.: US 2018/0207049 A1
(43) Pub. Date: Jul. 26, 2018

(54) Stretching Table and Method

(71) Applicant: ScoliWRx Inc., Campbell, CA (US)

(72) Inventor: Matthew I. Janzen, Campbell, CA (US)

(21) Appl. No.: 15/881,629

(22) Filed: Jan. 26, 2018

Related U.S. Application Data
(60) Provisional application No. 62/450,755, filed on Jan. 26, 2017.

(51) Int. Cl. A61H 1/02; A61F 5/058

(52) U.S. Cl. CPC A61H 1/0292; A61F 5/0585; A61H 2201/1215; A61H 2201/5064; A61H 2203/0468; A61H 2205/081; A61H 2201/5061; A61H 2201/1253

(57) ABSTRACT

A stretching platform includes a planar support structure having an upper surface in a substantially horizontal orientation. The planar support structure is configured to support a person lying in a prone position on the planar support structure and facing upward. A hip strap is configured to extend upward from the planar support structure and over hips of the person when the person is lying in the prone position on the planar support structure. A leg lifting mechanism is configured to lift a leg of the person when the person is lying in the prone position on the planar support structure and when hips of the person are secured by the hip strap in a substantially immobile manner relative to the planar support structure. The leg lifting mechanism is configured to apply an upward force to a posterior side of the leg at a location near a foot of the leg.

### CLAIM OF PRIORITY

This application claims priority under 35 U.S.C. 119(e) to U.S. Provisional Patent Application No. 62/450,755, filed Jan. 26, 2017, the disclosure of which is incorporated herein by reference in its entirety for all purposes.

### BACKGROUND

The field of invention is devices and methods that deploy such devices to correct spinal deformities, in particular spinal scoliosis. Braces and Schroth exercise routines are the standard conservative intervention in scoliosis. Further, braces and the German-developed Schroth exercises have been used separately and together to correct various types of curvatures of the spine, known as scoliosis. Some conditions cannot be fully corrected, and if they progress may necessitate spinal surgery procedures, including fusion and/or the implantation of medical devices to internally support the spine in a correct position. Hence, it is desirable to provide corrective methods to patients to avoid future surgical intervention which poses risks, expense and at least temporary absence of the patient from work and/or school. Accordingly, there is a need for improved non-surgical care, braces and exercise routines that more effectively prevent and arrest progressive scoliosis, as well as treat scoliosis. It is also of interest to have methods for scoliosis treatment that more effectively and rapidly reverse scoliosis once it has occurred and help patients return to a normal posture and flexibility.

### SUMMARY

In an example embodiment, a stretching platform includes a planar support structure having an upper surface in a substantially horizontal orientation. The planar support structure is configured to support a person lying in a prone position on the planar support structure and facing upward. A hip strap is configured to extend upward from the planar support structure and over hips of the person when the person is lying in the prone position on the planar support structure. A leg lifting mechanism is configured to lift a leg of the person when the person is lying in the prone position on the planar support structure and when hips of the person are secured by the hip strap in a substantially immobile manner relative to the planar support structure. The leg lifting mechanism is configured to apply an upward force to a posterior side of the leg at a location near a foot of the leg.

### BRIEF DESCRIPTION OF THE DRAWINGS

FIG. 1A shows a top view of a stretching platform, in accordance with some embodiments of the present invention.

FIG. 1B shows a vertical cross-section view of the stretching platform, corresponding to View A-A as referenced in FIG. 1A, in accordance with some embodiments of the present invention.

FIG. 1C shows a view toward a distal end of the stretching platform, corresponding to View B-B as referenced in FIG. 1A, in accordance with some embodiments of the present invention.

FIG. 1D shows a view toward a proximal end of the stretching platform, corresponding to View C-C as referenced in FIG. 1A, in accordance with some embodiments of the present invention.

FIG. 2A shows a top view of an example leg splint, in accordance with some embodiments of the present invention.

FIG. 2B shows a vertical cross-section view of the leg splint, corresponding to View A-A as referenced in FIG. 2A, in accordance with some embodiments of the present invention.

FIG. 2C shows a view toward the distal end of the leg splint, corresponding to View B-B as referenced in FIG. 2A, in accordance with some embodiments of the present invention.

FIG. 2D shows a view toward a proximal end of the leg splint, corresponding to View C-C as referenced in FIG. 2A, in accordance with some embodiments of the present invention.

FIG. 3 shows a vertical cross-section view of the stretching platform, corresponding to View D-D as referenced in FIG. 1A, with the person fitted within the stretching platform and engaging in stretching exercise, in accordance with some embodiments of the present invention.

FIG. 4 shows an isometric view of the stretching platform with the person fitted within the stretching platform and engaging in stretching exercise, in accordance with some embodiments of the present invention.

### DETAILED DESCRIPTION

In the following description, numerous specific details are set forth in order to provide an understanding of the present invention. It will be apparent, however, to one skilled in the art that the present invention may be practiced without some or all of these specific details. In other instances, well known process operations have not been described in detail in order not to unnecessarily obscure the present invention.

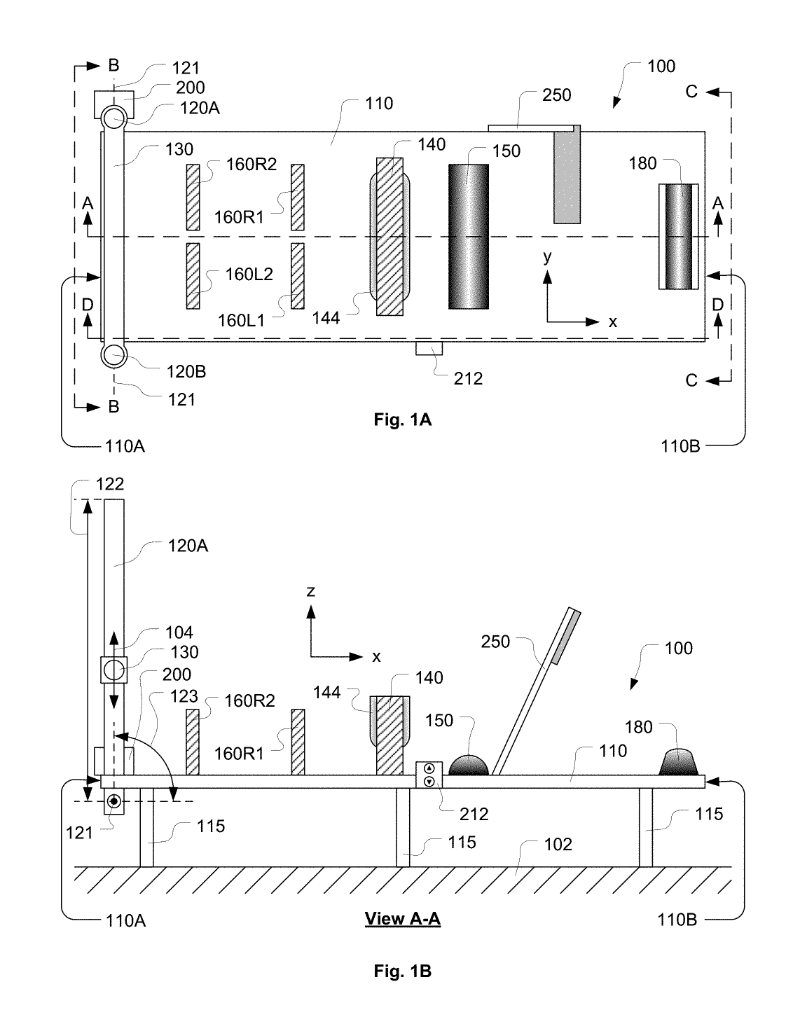

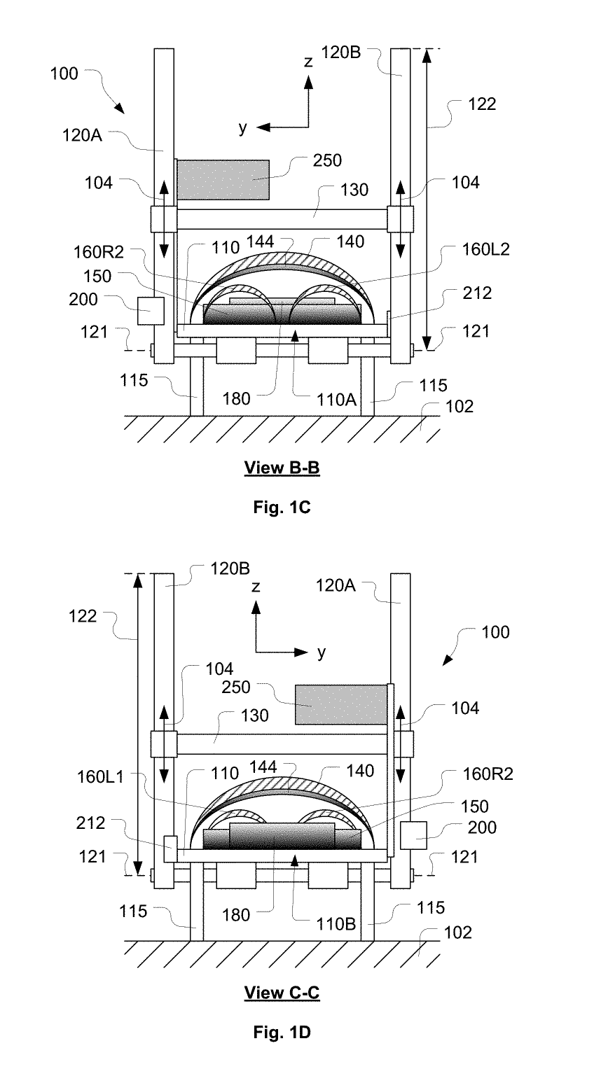

FIG. 1A shows a top view of a stretching platform 100, in accordance with some embodiments of the present invention. FIG. 1B shows a vertical cross-section view of the stretching platform 100, corresponding to View A-A as referenced in FIG. 1A, in accordance with some embodiments of the present invention. FIG. 1C shows a view toward a distal end 110A of the stretching platform 100, corresponding to View B-B as referenced in FIG. 1A, in accordance with some embodiments of the present invention. FIG. 1D shows a view toward a proximal end 110B of the stretching platform 100, corresponding to View C-C as referenced in FIG. 1A, in accordance with some embodiments of the present invention.

The stretching platform 100 includes a planar support structure 110 configured to support a person 1 in a prone position. In some embodiments, the planar support structure 110 is oriented in a substantially horizontal manner so that the planar support structure 110 provides a substantially level surface upon which the person 1 can lay down. In some embodiments, the planar support structure 110 is supported by a number of leg structures 115 at a vertical position above a floor 102 upon which the stretching platform 100 is positioned, where the vertical position is set so that it is easier for the person 1 to lay down on the planar support structure 110 and get up from the planar support structure 110. In some embodiments, vertical extents of the leg structures 115 are adjustable to provide for positioning of the planar support structure 110 at a prescribed angle relative to horizontal. In some embodiments, the vertical extents of the leg structures 115 are set so that the top surface of the planar support structure 110 upon which the person 1 lays down is substantially parallel with horizontal. In some embodiments, the vertical extents of the leg structures 115 are set so that the top surface of the planar support structure 110 upon which the person 1 lays down is not parallel with horizontal. For example, the vertical extents of the leg structures 115 can be set so that the proximal end 110B of the top surface of the planar support structure 110 is either higher or lower than the distal end 110A of the top surface of the planar support structure 110.

The stretching platform 100 also includes a hip strap 140 configured to extend in a transverse direction (y-direction) of the planar support structure 110 over and around the person 1, when the person 1 is laying down on the planar support structure 110. It should be understood that the transverse direction of the planar support structure 110 as referred to herein extends across the planar support structure 110 in a direction perpendicular to a longitudinal direction (x-direction) of the planar support structure 110, wherein the longitudinal direction extends perpendicularly between the distal end 110A and the proximal end 110B of the planar support structure 110. And, the vertical direction referred to herein is the z-direction or elevation direction above the floor 102. In some embodiments, the hip strap 140 is connected to the stretching platform 100 at a position near a middle region of the planar support structure 110 in the longitudinal direction (x-direction), such that the person 1 is able to lay down on the planar support structure 110 without hanging off of the planar support structure 110 with the hip strap 140 extending over the hip bones of the person 1. In some embodiments, the hip strap 140 is positioned to contact the person 1 at locations just below the iliac crests of the ilium bones of the pelvis of the person 1. The hip strap 140 can be tightened toward the planar support structure 110 and hence toward the person 1 to limit movement of the hips and buttocks and waist of the person 1 as the person 1 performs stretching exercises within the stretching platform 100. More specifically, the hip strap 140 can be tightened to prevent lifting of the hips and/or buttocks of the person 1 away from the planar support structure 110 as one or more legs of the person are raised upward away from the planar support structure 110, thereby enabling isolated stretching of spinal nerves. In some embodiments, a padding material 144 can be positioned between the hip strap 140 and the person 1 to improve comfort of the person 1 as the hip strap 140 is tightened. In some embodiments, the padding material 144 can be configured as a wide cushioned band.

It should be understood that in various embodiments, the hip strap 140 can be adjustable in length and shape to enable a proper fit to the person 1 and to accommodate variations in body sizes and shapes of different persons 1. Also, in some embodiments, the position of the hip strap 140 can be adjusted and/or moved in the longitudinal direction (x-direction) of the planar support structure 110. And, in some embodiments, the position of the hip strap 140 can be adjusted and/or moved in the transverse direction (y-direction) of the planar support structure 110. Additionally, in various embodiments, the hip strap 140 can be equipped with essentially any type of fastening and tightening mechanisms, such as a buckle, a fastener, a slide closure, a latch closure, a ratchet mechanism, a Velcro closure, among others. In various embodiments, the hip strap 140 can be connected to either a top surface, a side surface, and/or a bottom surface of the planar support structure 110. And, in some embodiments, the hip strap 140 can extend through the planar support structure 110, by way of slots formed through the planar support structure 110, to connect to the bottom surface of the planar support structure 110 or to connect to another structure located beneath the planar support structure 110.

In some embodiments, the stretching platform 100 can also include one or more leg retention straps 160L1, 160R1, 160L2, 160R2 for holding legs of the person 1 against the planar support structure 110. More specifically, in some embodiments, the leg retention straps 160L1 and/or 160L2 can be used to hold a left leg of the person 1 against the planar support structure 110. And, the leg retention straps 160R1 and/or 160R2 can be used to hold a right leg of the person 1 against the planar support structure 110. Each of the leg retention straps 160L1, 160R1, 160L2, 160R2 is configured to extend in the transverse direction (y-direction) of the planar support structure 110 over and around the corresponding leg of the person 1, when the person 1 is laying down on the planar support structure 110. In some embodiments, whichever leg of the person 1 is not being lifted by the stretching platform 100 during a given stretching exercise is instead strapped to the planar support structure 110 using a corresponding one or more of the leg retention straps 160L1, 160R1, 160L2, 160R2.

In some embodiments, the stretching platform 100 includes a single leg retention strap 160L1, 160R1 for each leg of the person 1. In some embodiments, the stretching platform 100 includes two leg retention straps 160L1, 160R1, 160L2, 160R2 for each leg of the person 1, i.e., 160L1 and 160L2 for the left leg and 160R1 and 160R2 for the right leg. In various embodiments, any of the leg retention straps 160L1, 160R1, 160L2, 160R2 can be adjustable in length and shape to enable a proper fit to the person 1 and to accommodate variations in body sizes and shapes of different persons 1. Also, in some embodiments, the position of any of the leg retention straps 160L1, 160R1, 160L2, 160R2 can be adjusted and/or moved in the longitudinal direction (x-direction) of the planar support structure 110. And, in some embodiments, the position of any of the leg retention straps 160L1, 160R1, 160L2, 160R2 can be adjusted and/or moved in the transverse direction (y-direction) of the planar support structure 110. Additionally, in various embodiments, any of the leg retention straps 160L1, 160R1, 160L2, 160R2 can be equipped with essentially any type of fastening and tightening mechanisms, such as a buckle, a fastener, a slide closure, a latch closure, a ratchet mechanism, a Velcro closure, among others. In various embodiments, any of the leg retention straps 160L1, 160R1, 160L2, 160R2 can be connected to either a top surface, a side surface, and/or a bottom surface of the planar support structure 110. And, in some embodiments, any of the leg retention straps 160L1, 160R1, 160L2, 160R2 can extend through the planar support structure 110, by way of slots formed through the planar support structure 110, to connect to the bottom surface of the planar support structure 110 or to connect to another structure located beneath the planar support structure 110.

In some embodiments, any of the leg retention straps 160L1, 160R1, 160L2, 160R2 can be tightened toward the planar support structure 110 and hence toward the person 1 to limit movement of one leg of the person 1 as the other leg of the person 1 is raised during performance of a stretching exercise within the stretching platform 100. In some embodiments, any of the leg retention straps 160L1, 160R1, 160L2, 160R2 can be equipped with padding material 144 to improve comfort of the person 1. In some embodiments, the leg retention straps 160L1, 160R1, 160L2, 160R2 are configured to remain loose around the leg(s) of the person 1, and function to allow some movement of the leg(s) of the person 1 while preventing significant upward movement of the leg(s) of the person 1. And, in some embodiments, the leg retention straps 160L1, 160R1, 160L2, 160R2 are either not present on the stretching platform 100 and/or are not utilized during stretching exercises.

By having multiple leg retention straps 160L1, 160R1, 160L2, 160R2 that are positioned in a spaced apart manner in the longitudinal direction (x-direction) of the planar support structure 110, the leg retention straps 160L1, 160R1, 160L2, 160R2 can be used on persons 1 of different height, and/or the leg retention straps 160L1, 160R1, 160L2, 160R2 can be positioned both above and below the knee(s) of the person 1, if desired. Also, in some embodiments, the stretching platform 100 can include a single leg retention strap (any of 160L1, 160R1, 160L2, 160R2, by way of example) that can be adjusted in position to hold either the left leg or right leg of the person 1.

The stretching platform 100 also includes a lumbar support pad 150 configured to extend across a portion of the planar support structure 110 in the transverse direction (y-direction) of the planar support structure 110. In various embodiments, a position of the lumbar support pad 150 is adjustable in the longitudinal direction (x-direction) and/or transverse direction (y-direction) of the planar support structure 110. In some embodiments, the position of the lumbar support pad 150 can be fixed during performance of stretching exercises using the stretching platform 100. In various embodiments, the lumbar support pad 150 can be fixed in position on the planar support structure 110 using essentially any type of fastening mechanisms, such as a strap(s), buckles, snaps, Velcro, among others. In various embodiments, the lumbar support pad 150 is formed of a partially compressible material that provides support to a lumbar region of the spinal column of the person 1 when the person 1 is lying down on their back on the planar support structure 110. In various embodiments, the partially compressible material of the lumbar support pad 150 can be one or more of foam, air, water, gel, gel filled elastic materials, fabric, pillow material, among others.

Also, in some embodiments, the stretching platform 100 can include a head support pad 180 disposed near the proximal end 110B of the planar support structure 110. The head support pad 180 can be configured to allow the person 1 to rest their head at an elevated angle above the planar support structure 110. In this manner, the head support pad 180 can help the person 1 view reading material and/or an electronic display, and enable the person 1 to have a better view of any optional levers, actuators, or other equipped associated with operation of the stretching platform 100. For example, in some embodiments, a tilted stand 250 can be connected to the stretching platform 100 to hold reading material and/or an electronic display to be viewed by the person 1, and the head support pad 180 can enable the person 1 to hold their head comfortably while looking toward the tilted stand 250.

The stretching platform 100 also includes a pair of spaced apart armatures 120A and 120B connected in rotary engagement with the distal end 110A of the planar support structure 110. The pair of spaced apart armatures 120A and 120B are configured to rotate together in a simultaneous and equivalent manner about an armature rotational axis 121, where the armature rotational axis 121 extends in the transverse direction (y-direction) of the planar support structure 110 and is located at the distal end 110A of the planar support structure 110. A first of the armatures 120A is positioned on a first side of the planar support structure 110. And, a second of the armatures 120B is positioned on a second side of the planar support structure 110. Each of the armatures 120A and 120B has an elongated shape extending from a first end to a second end, where the first end is located proximate to the armature rotational axis 121, and where the second end is located at a fixed distance 122 away from the armature rotational axis 121. The pair of spaced apart armatures 120A and 120B are configured and connected to co-rotate within a plane that is perpendicular to the top surface of the planar support structure 110 and parallel with the longitudinal direction (x-direction) of the planar support structure 110, i.e., within the z-x plane. In this manner, an angle of rotation 123 of the armatures 120A and 120B is measured in the z-x plane.

A lateral support arm 130 extends between the pair of spaced apart armatures 120A and 120B in the transverse direction (y-direction) of the planar support structure 110. A first end of the lateral support arm 130 is slidably-coupled to the first armature 120A, and a second end of the lateral support arm 130 is slidably-coupled to the second armature 120B. In this manner, the lateral support arm 130 is able to slide along the lengths of the pair of spaced apart armatures 120A and 120B, as indicated by the arrow 104, while an orientation of the lateral support arm 130 is maintained in the transverse direction (y-direction) of the planar support structure 110. In various embodiments, a controlled force is applied to move the lateral support arm 130 along the lengths of the pair of spaced apart armatures 120A and 120B. Therefore, it should be understood that a position of the lateral support arm 130 along the lengths of the pair of spaced apart armatures 120A and 120B can be carefully controlled. In some embodiments, the lateral support arm 130 can have tubular end fittings that fit over the armatures 120A and 120B. In some embodiments, the lateral support arm 130 can engage the armatures 120A and 120B using tracks and/or rails with corresponding slide fittings that fit into the tracks and/or rails and slide within the tracks and/or rails. In some embodiments, the lateral support arm 130 can engage the armatures 120A and 120B using flat gears.

During use of the stretching platform 100, the person 1 lies down on the planar support structure 110 in an upward facing orientation, with the hips of the person 1 substantially immobilized against the planar support structure 110 by the hip strap 140. A leg of the person 1 that is to be raised in the stretching exercise is placed in a leg splint 170. The leg splint 170 is configured to hold the leg of the person 1 is a substantially straight orientation so that the leg cannot bend at the knee, and with the corresponding foot of the person 1 pointed substantially upward. The leg splint 170 is configured to allow for upward rotation of the leg about the hip joint. Also, a leg of the person 1 that is not to be raised in the stretching exercise can be secured to the planar support structure 110 by one or more of the leg straps 160L1, 160R1, 160L2, 160R2. In some embodiments, the leg of the person 1 that is not to be raised in the stretching exercise can be held in a substantially straight orientation so that the leg cannot bend at the knee. In some embodiments, the leg of the person 1 that is not to be raised in the stretching exercise can be held in a bent orientation where the leg is bent at the knee.

During use of the stretching platform 100 in the above-described manner, resistance to further upward leg movement becomes clinically meaningful to provide improved exercise and treatment regimens when stretching spinal related nerves so to prevent and treat scoliosis caused by a retarded growth rate of the spinal nerves with respect to the growth of the bones in the spinal column. Therefore, it should be understood that the stretching platform 100 can be used to perform a process for stretching spinal nerves by positioning the person 1 on the platform, restraining the hips and one leg, and then repeatedly lifting the opposing, unrestrained leg. Also, it can be helpful to position the lumbar support pad 150 under the lumbar region of the spinal column of the person 1 to prevent straightening of the lumbar curve of the spinal column, which could provide an unwanted release mechanism for desired nerve tension during lifting of the leg.

During operation of the stretching platform 100, a distal end 170A of the leg splint 170 is positioned over the lateral support arm 130. In some embodiments, the pair of spaced apart armatures 120A and 120B are rotated to enable positioning of the lateral support arm 130 at a starting rest position with the leg splint 170 is positioned over the lateral support arm 130. In some embodiments, the pair of spaced apart armatures 120A and 120B extend in a substantially vertical direction (z-direction) at the starting rest position. Then, during the stretching exercise, the lateral support arm 130 is moved in a controlled manner along the lengths of the pair of spaced apart armatures 120A and 120B, with the pair of spaced apart armatures 120A and 120B allowed to freely rotate about the armature rotational axis 121. As the lateral support arm 130 is moved in the controlled manner along the lengths of the pair of spaced apart armatures 120A and 120B, the distal end 170A of the leg splint 170 will be raised in a controlled manner by the lateral support arm 130. In some embodiments, the lateral support arm 130 is engaged with the leg splint 170 so that the lateral support arm 130 can rotate freely relative to the leg splint 170 while being confined to a fixed position along the length of the leg splint 170. During the stretching exercise, the lateral support arm 130 can be held at any location along the lengths of the pair of spaced apart armatures 120A and 120B to correspondingly hold the distal end 170A of the leg splint 170 at a desired vertical distance above the planar support structure 110.

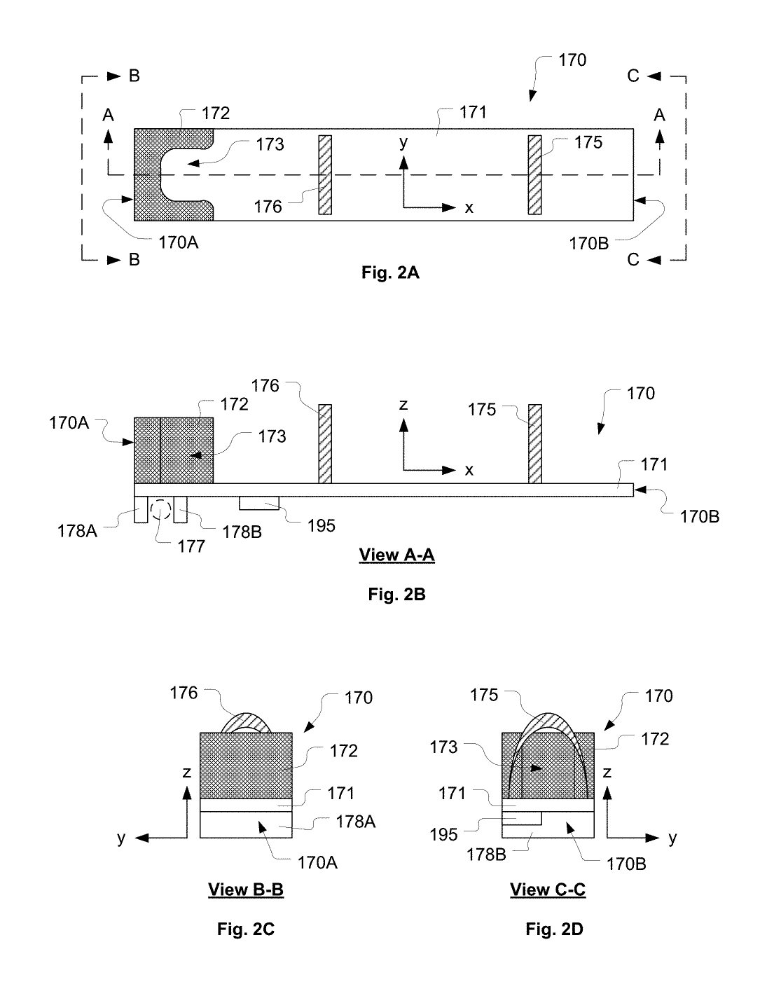

FIG. 2A shows a top view of an example leg splint 170, in accordance with some embodiments of the present invention. FIG. 2B shows a vertical cross-section view of the leg splint 170, corresponding to View A-A as referenced in FIG. 2A, in accordance with some embodiments of the present invention. FIG. 2C shows a view toward the distal end 170A of the leg splint 170, corresponding to View B-B as referenced in FIG. 2A, in accordance with some embodiments of the present invention. FIG. 2D shows a view toward a proximal end 170B of the leg splint 170, corresponding to View C-C as referenced in FIG. 2A, in accordance with some embodiments of the present invention. The leg splint 170 is configured to support the foot and lower calf portion of the leg that keeps the ankle joint and knee joint generally immobile, while allowing the leg to pivot/rotate at the hip joint as the lateral support arm 130 is moved along the lengths of the pair of spaced apart armatures 120A and 120B. Such pivoting/rotating of the leg at the hip joint while holding the ankle joint and knee joint in a generally immobile state provides multiple benefits, including stretching of spinal nerves that extend downward into the leg, as well as stretching of muscles and ligaments, such as hamstring ligaments. For treatment of scoliosis, stretching of the nerves that extend downward into the leg is a primary therapeutic objective.

In the example embodiment of FIGS. 2A-2D, the leg splint 170 includes a planar support 171 configured as a substantially rigid structure. In various embodiments, the planar support 171 is a rigid elongated member having a generally planar shape. In some embodiments, the planar support 171 can have an upper surface of substantially flat shape. In some embodiments, the planar support 171 can have an upper surface that is contoured to substantially match an exterior contour (posterior contour) of the leg and foot of the person 1. In some embodiments, the upper surface of the planar support 171 can be padded to conform to the exterior contour (posterior contour) of the leg and foot of the person 1. In various embodiments, the planar support 171 can include a cast, a boot, a boot-type cast, a restraint, a ski boot type component, or other component that provides for securing of the leg of the person 1 to the planar support 171 and that provides for immobilization of the ankle of the leg of the person 1. In some embodiments, an upright upper protuberance 172 is positioned on the upper surface of the planar support 171 at the distal end 170A of the planar support 171. In some embodiments, the upright upper protuberance 172 includes a recess 173 configured to receive a heel and/or shoe within which the foot of the person 1 is present. In some embodiments, the recess 173 is configured to receive a bare foot or sock covered foot of the person 1. In various embodiments, the upright upper protuberance 172 is formed of a compliant material and/or padded rigid material to provide comfort to the person 1 while also providing sufficient mechanical strength to securely hold the foot of the person 1.

In some embodiments, the leg splint 170 includes a pair of spaced apart adjustable straps 175 and 176 that extend across the planar support 171 so as to extend over and around the leg of the person 1 to hold the leg of the person 1 against the planar support 171. In some embodiments, the strap 175 is positioned to extend over the leg of the person 1 at a location between the knee and foot of the person 1. And, in some embodiments, the strap 176 is positioned to extend over the leg of the person 1 at a location between the knee and hip of the person 1. In some embodiments, the positions of the straps 175 and 176 along the length of the planar support 171 are adjustable. In various embodiments, the straps 175 and 176 can be equipped with essentially any type of fastening and tightening mechanisms, such as a buckle, a fastener, a slide closure, a latch closure, a ratchet mechanism, a Velcro closure, among others. In various embodiments, the straps 175 and 176 can be connected to either a top surface, a side surface, and/or a bottom surface of the planar support 171. And, in some embodiments, the straps 175 and 176 can extend through the planar support 171, by way of slots formed through the planar support 171, to connect to the bottom surface of the planar support 171 or to connect to another structure located beneath the planar support 171.

In various embodiments, the leg splint 170 includes a means for hinged or rotary engagement with the lateral support arm 130. In some embodiments, a pair of spaced apart blocks 178A and 1788 are connected to the lower surface of the planar support 171 at a location near the distal end 170A of the planar support 171. The blocks 178A and 1788 are spaced apart along the length of the leg splint 170 to form a cavity 177 between the blocks 178A and 1788 and below the planar support 171. The blocks 178A and 1788 are shaped so that the cavity 177 extend across the leg splint 170 in a direction substantially parallel with the direction of extent of the lateral support arm 130. In this manner, the cavity 177 is sized and shaped to receive the lateral support arm 130 and restrict movement of the lateral support arm 130 along the length of the leg splint 170, while also providing for free rotation of the lateral support arm 130 within the cavity 177 relative to the lateral support arm 130, as the lateral support arm 130 is moved along the lengths of the pair of spaced apart armatures 120A and 120B to raise and lower the distal end 170A of the planar support 171. It should be understood that in various embodiments, different mechanisms other than the blocks 178A and 1788 can be used to restrict movement of the lateral support arm 130 along the length of the leg splint 170, while also providing for free rotation of the lateral support arm 130 relative to the lateral support arm 130, such as one or more of hinges, swivels, rotary joints, couplings, among others. Also, it should be understood that with the blocks 178A and 1788 and/or other mechanisms for engaging the lateral support arm 130 with the planar support 171, the lateral support arm 130 can be quickly disconnected or disengaged from the planar support 171.

In various embodiments, controlled movement of the lateral support arm 130 along the lengths of the pair of spaced apart armatures 120A and 120B can be done by applying manual force and/or with mechanical assistance 200. In various embodiments, components to assist with movement of the lateral support arm 130 along the lengths of the pair of spaced apart armatures 120A and 120B can include ratchets, pulleys, ropes, belts, chains, electric motor(s), gears, mechanical linkage(s), or essentially any other mechanical component that is configurable to assist with application of force to the lateral support arm 130.

In some embodiments, the mechanical assistance 200 can include one or more lever arm(s), and/or one or more motor(s), and/or corresponding mechanical linkage/gears connected an actuator 212 than can be reached and operated by the person 1. The person 1 can control the actuator 212 to control operation of the mechanical assistance 200 to cause controlled movement of the lateral support arm 130 along the lengths of the pair of spaced apart armatures 120A and 120B. In some embodiments, the control of the actuator 212 directs the mechanical assistance 200 to apply force to the lateral support arm 130 without electro-mechanical assistance, i.e., without operation of an electric motor. In some embodiments, the control of the actuator 212 directs the mechanical assistance 200 to operate one or more electric motor(s) to apply force to the lateral support arm 130. In various embodiments, the actuator 212 can be operated by the person 1 that is being stretched or by as assistant. In various embodiments, the mechanical assistance 200 can be equipped with one or more force sensors or displacement sensors to control and/or limit an amount of force applied to the lateral support arm 130. Also, in various embodiments, the mechanical assistance 200 can include a manual release mechanism to enable disengagement of any motor or other equipment used to apply force to the lateral support arm 130, particularly in the event of an emergency. Also, in various embodiments, stops and/or ratchet mechanisms can be installed between one or more of the armatures 120A, 120B and the planar support structure 110 to keep the armatures 120A, 120B at a fixed vertical position above the planar support structure 110 while the person 1 is entering and exiting the stretching platform 100 and being fitted to the stretching platform 100.

In some embodiments, a position sensor 195, such as an accelerometer and/or tilt monitor, can be disposed on the leg splint 170 and/or on the lateral support arm 130 to measure, record, and provide data on the position of the leg of the person 1 as a function of time. In some embodiments, the position sensor 195 can be used to count each step of repeated leg flexing, time and day of use, as well as the angle of the hip joint at each instance of raising the leg. In some embodiments, the mechanical assistance 200 can be controlled in an automatic manner through programmed instructions executed by a computer system 300, where the computer system 300 can be essentially any type of computing device, such as a smartphone, a tablet computer, a laptop computer, a desktop computer, a programmable logic controller (PLC), or other type of computing device that includes a microprocessor and memory. And, data measured by the position sensor 195 can be provided to the computer system 300 through wired and/or wireless data communication methods, so that the data can be processed by the computer system 300 and acted upon by the computer system 300. In some embodiments, the computer system 300 is configured to implement a closed-loop feedback process using the data measured by the position sensor 195 in order to operate the stretching platform 100 to execute a prescribed stretching routine on the person 1. The computer system 300 can also be configured to log usage of the stretching platform 100 and track progress of the person 1 toward prescribed stretching goals.

In some embodiments, programmed automatic control of the mechanical assistance 200 by the computer system 300 can provide for timed constant or cyclic application of angular displacement of the armatures 120A and 120B and/or movement of the lateral support arm 130 along the armatures 120A and 120B. Instructions, such as a duty cycle, for a stretching exercise can be programmed into the computer system 300 or transmitted to the computer system 300 from another device, such as from a portable computing device.

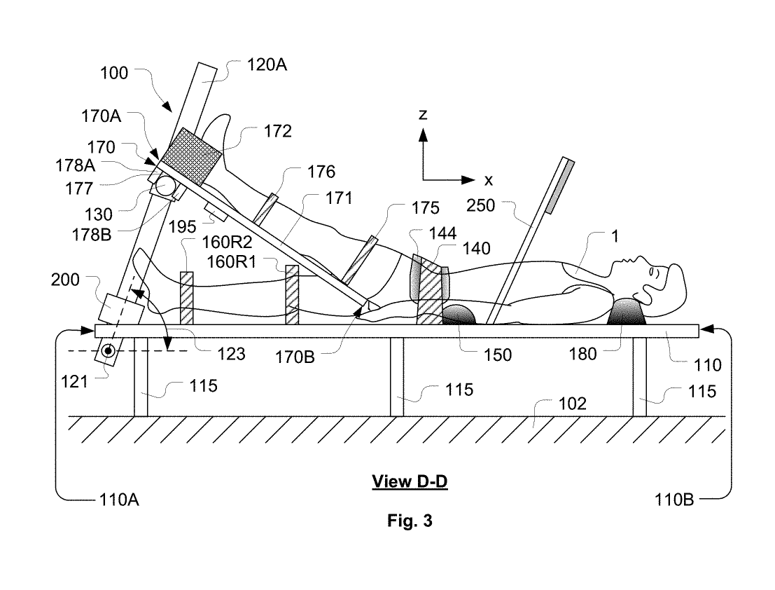

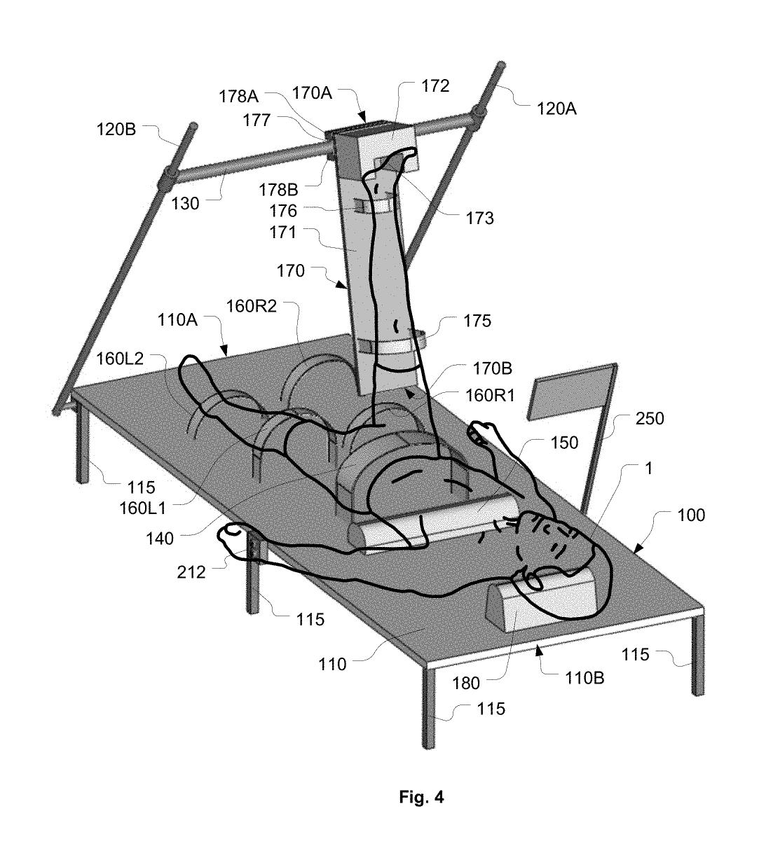

FIG. 3 shows a vertical cross-section view of the stretching platform 100, corresponding to View D-D as referenced in FIG. 1A, with the person 1 fitted within the stretching platform 100 and engaging in stretching exercise, in accordance with some embodiments of the present invention. FIG. 4 shows an isometric view of the stretching platform 100 with the person 1 fitted within the stretching platform 100 and engaging in stretching exercise, in accordance with some embodiments of the present invention.

The stretching platform 100 as disclosed herein includes the planar support structure 110 having an upper surface in a substantially horizontal orientation. The planar support structure 110 is configured to support the person 1 lying in a prone position on the planar support structure 110 and facing upward. The hip strap 140 is configured to extend upward from the planar support structure 110 and over hips of the person 1 when the person 1 is lying in the prone position on the planar support structure 110. In some embodiments, the padding material 144 is connected to the hip strap 140 at a location between the hip strap 140 and the planar support structure 110. A leg lifting mechanism is configured to lift a leg of the person 1 when the person 1 is lying in the prone position on the planar support structure 110 and when hips of the person 1 are secured by the hip strap 140 in a substantially immobile manner relative to the planar support structure 110. The leg lifting mechanism is configured to apply an upward force to a posterior side of the leg at a location near a foot of the leg. In some embodiments, the lumbar support pad 150 is positioned on the planar support structure 110 at a location beneath a lumbar portion of a spinal column of the person 1 when the person 1 is lying in the prone position on the planar support structure 110. In some embodiments, the lumbar support pad 150 is secured to the planar support structure 110. In some embodiments, a position of the lumbar support pad 150 on the planar support structure 110 is adjustable.

The leg of the person that is to be lifted by the leg lifting mechanism can be considered a first leg. In some embodiments, the stretching platform 100 includes one or more of the leg retention straps 160L1, 160R1, 160L2, 160R2 configured to extend upward from the planar support structure 110 and over a second leg of the person 1 when the person 1 is lying in the prone position on the planar support structure 110. The one or more of the leg retention straps 160L1, 160R1, 160L2, 160R2 is/are configured to secure the second leg of the person in a substantially immobile manner relative to the planar support structure 110. In some embodiments, a position of the one or more of the leg retention straps 160L1, 160R1, 160L2, 160R2 on the planar support structure 110 is/are adjustable. In some embodiments, a first leg retention strap 160L1, 160R1 is positioned on the planar support structure 110 to extend over the second leg of the person at a location between a knee of the second leg and hips of the person 1 when the person 1 is lying in the prone position on the planar support structure 110, and a second leg retention strap 160L2, 160R2 is positioned on the planar support structure 110 to extend over the second leg of the person 1 at a location between the knee and a foot of the second leg when the person 1 is lying in the prone position on the planar support structure 110.

In some embodiments, the stretching platform 100 includes the head support pad 180 positioned on the planar support structure 110 at a location beneath a head of the person 1 when the person 1 is lying in the prone position on the planar support structure 110. In some embodiments, the head support pad 180 is secured to the planar support structure 110. In some embodiments, a position of the head support pad 180 on the planar support structure 110 is adjustable. In some embodiments, the tilted stand 250 is connected to the planar support structure 110, where the tilted stand 250 is configured to hold an item for viewing by the person 1 when the person 1 is lying in the prone position on the planar support structure 110. In some embodiments, a position of the tilted stand 250 relative to the planar support structure 110 is adjustable.

In some embodiments, the leg lifting mechanism includes the lateral support arm 130 that extends beneath the posterior side of the leg (to be lifted) at the location near the foot of the leg when the person 1 is lying in the prone position on the planar support structure 110. The leg lifting mechanism is configured to lift the lateral support arm 130 in a controlled manner above the planar support structure 110 while substantially maintaining a position of the lateral support arm 130 at the location near the foot of the leg. In some embodiments, the leg lifting mechanism includes a manually operated lifting device configured and connected to provide controlled lifting of the lateral support arm 130 above the planar support structure 110. In some embodiments, the manually operated lifting device does not include an electric motor. In some embodiments, the leg lifting mechanism includes an electric motor configured and connected to provide controlled lifting of the lateral support arm 130 above the planar support structure 110. In some embodiments, one or more sensors is/are configured and connected to measure a position of the lateral support arm 130 above the planar support structure 110. In some embodiments, the lateral support arm 130 is configured to engage with the leg splint 170, where the leg splint 170 is configured to hold the leg (to be lifted) in a substantially straight configuration.

In some embodiments, the leg lifting mechanism includes a pair of armatures 120A, 120B connected in rotary engagement with the planar support structure 110 near the distal end 110A of the planar support structure 110. The distal end 110A of the planar support structure 110 is proximate to the foot of the leg (to be lifted) when the person 1 is lying in the prone position on the planar support structure 110. The pair of armatures 120A, 120B is configured to rotate together in a simultaneous and equivalent manner about the armature rotational axis 121. The armature rotational axis 121 extends in the transverse direction (y-direction) of the planar support structure 110. Each armature 120A, 120B of the pair of armatures 120A, 120B has an elongated shape extending from a first end to a second end, where the first end is located proximate to the armature rotational axis 121, and where the second end is located at a fixed distance away from the armature rotational axis 121. The lateral support arm 130 extends between the pair of armatures 120A, 120B in the transverse direction (y-direction) of the planar support structure 110. A first end of the lateral support arm 130 is slidably-coupled to a first armature 120A of the pair of armatures 120A, 120B, and a second end of the lateral support arm 130 is slidably-coupled to a second armature 120B of the pair of armatures 120A, 120B. In some embodiments, the first armature 120A of the pair of armatures 120A, 120B is positioned on a first side of the planar support structure 110, and the second armature 120B of the pair of armatures 120A, 120B is positioned on a second side of the planar support structure 110. In some embodiments, the pair of armatures 120A, 120B is configured to rotate freely about the armature rotational axis 121 as the lateral support arm 130 is moved along the elongated shape of the pair of armatures 120A, 120B.

In some embodiments, the leg lifting mechanism includes a manually operated lifting device configured and connected to provide controlled movement of the lateral support arm 130 along the elongated shape of the pair of armatures 120A, 120B. In some embodiments, the manually operated lifting device does not include an electric motor. In some embodiments, the leg lifting mechanism includes an electric motor configured and connected to provide controlled movement of the lateral support arm 130 along the elongated shape of the pair of armatures 120A, 120B. In some embodiments, one or more sensors is/are configured and connected to measure a position of the lateral support arm 130 along the elongated shape of the pair of armatures 120A, 120B. In some embodiments, the lateral support arm 130 is configured to engage with the leg splint 170 when the lateral support arm 130 is moved along the elongated shape of the pair of armatures 120A, 120B, where the leg splint 170 is configured to hold the leg (to be lifted) in a substantially straight configuration.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in other embodiments, even if not specifically shown or described. Such variations of the example embodiments disclosed herein are not to be regarded as a departure from the invention, and all such modifications are intended to be included within the scope of the invention.

Although the foregoing invention has been described in some detail for purposes of clarity of understanding, it will be apparent that certain changes and modifications can be practiced within the scope of the invention description. Accordingly, the example embodiments disclosed herein are to be considered as illustrative and not restrictive, and the invention is not to be limited to the details given herein, but may be modified within the scope and equivalents of the described embodiments.

What is claimed is:

1. A stretching platform, comprising:
a planar support structure having an upper surface in a substantially horizontal orientation, the planar support structure configured to support a person lying in a prone position on the planar support structure and facing upward;
a hip strap configured to extend upward from the planar support structure and over hips of the person when the person is lying in the prone position on the planar support structure; and
a leg lifting mechanism configured to lift a leg of the person when the person is lying in the prone position on the planar support structure and when hips of the person are secured by the hip strap in a substantially immobile manner relative to the planar support structure, the leg lifting mechanism configured to apply an upward force to a posterior side of the leg at a location near a foot of the leg.

2. The stretching platform as recited in claim 1, further comprising:
a lumbar support pad positioned on the planar support structure at a location beneath a lumbar portion of a spinal column of the person when the person is lying in the prone position on the planar support structure.

3. The stretching platform as recited in claim 2, wherein the lumbar support pad is secured to the planar support structure.

4. The stretching platform as recited in claim 2, wherein a position of the lumbar support pad on the planar support structure is adjustable.

5. The stretching platform as recited in claim 1, wherein the leg of the person that is to be lifted by the leg lifting mechanism is a first leg, the stretching platform including a leg retention strap configured to extend upward from the planar support structure and over a second leg of the person when the person is lying in the prone position on the planar support structure, the leg retention strap configured to secure the second leg of the person in a substantially immobile manner relative to the planar support structure.

6. The stretching platform as recited in claim 5, wherein a position of the leg retention strap on the planar support structure is adjustable.

7. The stretching platform as recited in claim 5, wherein the leg retention strap is a first leg retention strap, the stretching platform including a second leg retention strap configured to extend upward from the planar support structure and over the second leg of the person when the person is lying in the prone position on the planar support structure, the second leg retention strap configured to secure the second leg of the person in the substantially immobile manner relative to the planar support structure.

8. The stretching platform as recited in claim 7, wherein the first leg retention strap is positioned on the planar support structure to extend over the second leg of the person at a location between a knee of the second leg and hips of the person when the person is lying in the prone position on the planar support structure, and wherein the second leg retention strap is positioned on the planar support structure to extend over the second leg of the person at a location between the knee and a foot of the second leg when the person is lying in the prone position on the planar support structure.

9. The stretching platform as recited in claim 7, wherein a position of the first leg retention strap on the planar support structure is adjustable, and wherein a position of the second leg retention strap on the planar support structure is adjustable.

10. The stretching platform as recited in claim 1, further comprising:
a padding material connected to the hip strap at a location between the hip strap and the planar support structure.

11. The stretching platform as recited in claim 1, further comprising:
a head support pad positioned on the planar support structure at a location beneath a head of the person when the person is lying in the prone position on the planar support structure.

12. The stretching platform as recited in claim 11, wherein the head support pad is secured to the planar support structure.

13. The stretching platform as recited in claim 11, wherein a position of the head support pad on the planar support structure is adjustable.

14. The stretching platform as recited in claim 1, further comprising:
a tilted stand connected to the planar support structure, the tilted stand configured to hold an item for viewing by the person when the person is lying in the prone position on the planar support structure.

15. The stretching platform as recited in claim 14, wherein a position of the tilted stand relative to the planar support structure is adjustable.

16. The stretching platform as recited in claim 1, wherein the leg lifting mechanism includes a lateral support arm that extends beneath the posterior side of the leg at the location near the foot of the leg when the person is lying in the prone position on the planar support structure, wherein the leg lifting mechanism is configured to lift the lateral support arm in a controlled manner above the planar support structure while substantially maintaining a position of the lateral support arm at the location near the foot of the leg.

17. The stretching platform as recited in claim 16, wherein the leg lifting mechanism includes a manually operated lifting device configured and connected to provide controlled lifting of the lateral support arm above the planar support structure.

18. The stretching platform as recited in claim 17, wherein the manually operated lifting device does not include an electric motor.

19. The stretching platform as recited in claim 16, wherein the leg lifting mechanism includes an electric motor configured and connected to provide controlled lifting of the lateral support arm above the planar support structure.

20. The stretching platform as recited in claim 16, further comprising:
one or more sensors configured and connected to measure a position of the lateral support arm above the planar support structure.

21. The stretching platform as recited in claim 16, wherein the lateral support arm is configured to engage with a leg splint, wherein the leg splint is configured to hold the leg in a substantially straight configuration.

22. The stretching platform as recited in claim 16, wherein the leg lifting mechanism includes a pair of armatures connected in rotary engagement with the planar support structure near a distal end of the planar support structure, the distal end of the planar support structure proximate to the foot of the leg when the person is lying in the prone position on the planar support structure, the pair of armatures configured to rotate together in a simultaneous and equivalent manner about an armature rotational axis, wherein the armature rotational axis extends in a transverse direction of the planar support structure, wherein each armature of the pair of armatures has an elongated shape extending from a first end to a second end, where the first end is located proximate to the armature rotational axis, and where the second end is located at a fixed distance away from the armature rotational axis, wherein the lateral support arm extends between the pair of armatures in the transverse direction of the planar support structure, wherein a first end of the lateral support arm is slidably-coupled to a first armature of the pair of armatures, and wherein a second end of the lateral support arm is slidably-coupled to a second armature of the pair of armatures.

23. The stretching platform as recited in claim 22, wherein the first armature of the pair of armatures is positioned on a first side of the planar support structure, and wherein the second armature of the pair of armatures is positioned on a second side of the planar support structure.

24. The stretching platform as recited in claim 22, wherein the pair of armatures are configured to rotate freely about the armature rotational axis as the lateral support arm is moved along the elongated shape of the pair of armatures.

25. The stretching platform as recited in claim 24, wherein the leg lifting mechanism includes a manually operated lifting device configured and connected to provide controlled movement of the lateral support arm along the elongated shape of the pair of armatures.

26. The stretching platform as recited in claim 25, wherein the manually operated lifting device does not include an electric motor.

27. The stretching platform as recited in claim 24, wherein the leg lifting mechanism includes an electric motor configured and connected to provide controlled movement of the lateral support arm along the elongated shape of the pair of armatures.

28. The stretching platform as recited in claim 22, further comprising:
one or more sensors configured and connected to measure a position of the lateral support arm along the elongated shape of the pair of armatures.

29. The stretching platform as recited in claim 22, wherein the lateral support arm is configured to engage with a leg splint, wherein the leg splint is configured to hold the leg in a substantially straight configuration.
